(19) United States
(12) Patent Application Publication
(10) Pub. No.: US 2020/0146692 A1
(43) Pub. Date: May 14, 2020

(54) TOURNIQUET AND TISSUE PRESERVATION SYSTEM

(71) Applicant: University of Strathclyde, Glasgow (GB)

(72) Inventor: Terence Gourlay, Glasgow (GB)

(21) Appl. No.: 16/612,841

(22) PCT Filed: May 9, 2018
(86) PCT No.: PCT/GB2018/051240

§ 371 Date: Nov. 12, 2019

(30) Foreign Application Priority Data
May 12, 2017 (GB) 1707663.9

(51) Int. Cl. A61B 17/135; A61F 7/02

(52) U.S. Cl. CPC A61B 17/135; A61F 2007/0056; A61F 7/02

(57) ABSTRACT

A tourniquet apparatus (10) comprises at least two expandable members (21,22) configured for applying pressure on a limb of a subject; and actuating means (143,144) for actuating the at least two expandable members (21,22), wherein the actuating means (143,144) are configured to actuate each expandable member independently and/or selectively. There is also provided a limb-cooling apparatus (200) comprising an inner stretchable layer (210) configured to engage and/or contact a limb of a subject; an outer layer (220) at least partially attached to the inner layer (210); and a cooling system (240) configured to supply a cooling medium (250) between the inner layer (210) and the outer layer (220).

### FIELD OF INVENTION

The present invention relates to an apparatus, systems and methods for preserving a limb of a subject. In particular, though not exclusively, the present invention relates to a tourniquet apparatus, and to apparatuses and methods for improving preservation of an injured limb.

### BACKGROUND TO INVENTION

Preservation of a person's injured extremities or limbs is an important area of medical research. One common environment leading to injuries to a person's extremities or limbs in the military environment. Combat casualty mortality, defined as individuals who were killed in action and those who subsequently succumbed to wounds, is mainly the result of vascular trauma and haemorrhage (Kelly et al, 2008). However, approximately 25% of military personnel who die from explosion or gunshots have potentially survivable injuries (Eastridge et al, 2013) and thus many casualties rely on effective prehospital trauma care.

Data on potentially survivable deaths from the Vietnam War showed that 60% were from extremity haemorrhage exsanguination, 33% from tension pneumothorax, and 7% from airway obstruction (Maughon, 1970; Holcomb et al 2007; Kelly et al 2008; Bellamy 1984). Since then, improvements in prehospital care have taken precedence in military medicine to improve survival from combat injury. As such, battlefield survival of US casualties has significantly improved, in that a greater percentage of wounded soldiers survived during Operations Enduring and Iraqi Freedom (OEF and OIF) than in any other previous conflict (Starnes et al, 2006). This is due to lifesaving technologies such as improved body armour and improved patient resuscitation (Kragh et al, 2009; Ennis et al, 2008; Holcomb et al 2008; Eastridge et al 2006). As a result, survivable injury severity has significantly increased, and data collected from 2003 to 2006 in OIF indicate that the annual proportion of severe extremity injuries of US casualties rose from 21% to 44% (Kelly et al 2008).

Uncontrolled external haemorrhage of the extremities (also known as compressible bleeding) still remains the primary cause of preventable deaths in the field (Heldenberg et al 2015). Mortality rates of up to 9% have been recorded as being a result of poorly treated external haemorrhage (Starnes et al 2006; Mabry et al, 2000). During OIF, extremity vascular injuries accounted for 50-70% of all injuries, and exsanguination from these injuries is the leading cause of preventable death on the modern battlefield (Starnes et al 2006).

British and US Army battlefield analysis has shown that approximately 50% of injuries were to the extremities during OIF (Belmont et al 2010; Ramalingam 2004). Improvements in personal protective equipment (PPE) have considerably increased likelihood of survival in Iraq and Afghanistan (Nelson et al 2008; Galarneau et al 2008). Technological advances include body armour with blast resistant ballistic goggles or glasses worn with improved Kevlar Advanced Combat Helmets, although these do not protect the extremities (Hildreth, 2009; Nyein et al, 2010) in common contemporary situations where blast forces are a huge risk to the vulnerable lower extremities from detonated ground level IEDs, or where they are directed upward through the floor of a vehicle (Fox et al, 2005). Low intensity conflict in an urban environment is a modern battlefield phenomenon (as in OEF and OIF) which exposes soldiers to urban warfare and against weapons such as grenades, mines and IEDs (Starnes et al 2006; Husum et al 2004). This environment, coupled with personal armour that focuses on torso, head and neck protection, is a dominant cause of the sharp increase in the proportion of extremity injuries (Gofrit et al 2005; Gondusky & Reiter, 2005).

For allied military personnel in the Iraq and Afghanistan conflicts, explosive devices were the mechanism of injury associated with most (87.9%) amputations (Stansbury et al 2008). In Iraq alone, 1,158 US military personnel suffered limb amputations (Fischer, 2010). In the Iraq and Afghanistan wars, a military tactic from opposition forces was the placement of IEDs which increased the incidence of ‘dismounted IED injuries’—these leg and genital wounds resulted in approximately 6% of seriously wounded soldiers receiving lower limb amputations in the first 7 years. The rate of blast injuries has increased in both US and UK casualties since 2010 in Afghanistan and often involved multiple limb loss (Wallace, 2012) and the constellation of lower limb amputations has been dubbed the ‘new signature wound of the war’ (Brown, 2011), a term previously used in relation to mild traumatic brain injury (Okie, 2005).

In Afghanistan 2010, the rate of amputations increased whilst US combat deaths declined, from 437 to 368, and this further confirmed improvements in wound survivability (Carroll, 2012). Furthermore, in 2010-11, nearly half a sample of US combat fatalities suffered bilateral lower limb extremity amputations, with almost a third losing three limbs (Dismounted Complex Blast Injury Task Force, 2011). The socioeconomical impact of extremity injuries from OEF and OIF was considerable, in which this subset of casualties accounted for almost 65 percent of inpatient care (responsible for the most operating room time use, the largest the number of surgeries performed and the number of occupied beds (Cho et al 2005)), caused the greatest number of disabled soldiers, and resulted in the greatest projected disability costs (Masini et al 2009).

One well-known means of controlling potentially life-threatening extremity hemorrhage exsanguination, particularly in situations where the subject is not in proximity to any medical facilities, the use of tourniquets. The frequent use of tourniquets is the most obvious difference in practice between combat and civilian casualty care (Beekley et al 2008), and is recommended for use before patient extraction and transport (Kragh et al 2009). In a battlefield situation, haemorrhage control—and particularly tourniquet use—has been shown to significantly improve combat casualty survival, whilst poor tourniquet application has been associated with failure to control haemorrhage and death (Beekley et al 2008; Kragh et al 2009).

Analysis on potentially preventable deaths of Special Operations personnel showed a 15% incidence in Iraq and Afghanistan from the start until November 2004, and the authors commented that 25% of fatalities with potentially survivable injuries may have been saved with the simple application of a tourniquet (Holcomb et al, 2007; Kelly et al, 2008). A study of OIF casualties administered with a prehospital tourniquet showed significantly improved haemorrhage control, and that 57% of the deaths might have been prevented by earlier tourniquet use (Beekley et al 2008). Tourniquets were considered a last resort for extremity trauma and perceived as a resignation that limb amputation was imminent. Studies have, however, refuted this (Klenerman, 2003; Lakstein et al 2003) and led the United States Army Institute of Surgical Research (USAISR) to adopt tourniquet use as a standard and effective first-line haemorrhage control measure. The American experience in OIF and OEF was the major initiative for a breakthrough in tourniquet technology.

Mortality from isolated limb exsanguination during the course of OIF and OEF has dropped from 7 percent to 2 percent, and is credited with improved resuscitation protocols, better battlefield bandages, and more frequent use of tourniquets in the prehospital setting (Kelly et al 2008; Kragh et al 2009; Holcomb et al 2008).

Rapid application of a tourniquet in the prehospital setting has been shown to save 11% more lives than delayed use when the casualty reaches a hospital (Kragh et al 2009), and tourniquet placement before shock onset saves about 20-fold more casualties than placement after shock onset (96 percent survival with use before onset of shock versus 4 percent survival after onset of shock) (Kragh et al 2012). Often this procedure, whilst saving the life of the casualty, results in the amputation of the effected limb due to prolonged periods of ischaemia associated with the time involved in isolating the limb and transporting the casualty from the field with a tourniquet applied, and in some instances with the magnitude of the injury. The presence of haemorrhagic shock—which is highly likely in battlefield casualties—may compound the effect of extremity ischaemia and reduce the ischaemic threshold of the limb to less than 3 hours. It has been shown in large animal models that full limb neuromuscular recovery was only evident with ischaemia times <60 minutes in the setting of shock, and that attempts of restoration of flow should take place before irreversible injury (<3 hours) (Hancock et al, 2011).

However, one major problem associated with conventional tourniquets if that the prolonged use of such tourniquets often causes localised damage to the subject's tissue in the area where the tourniquet is applied.

The development of microsurgical techniques has allowed successful replantation surgery to become routine in military plastic surgical interventions. The incidence of tissue repair success is strongly correlated with the extent and duration of ischaemic injury—this is particularly critical when addressing major limb replantation or revascularisation. Skeletal muscle is the least tolerant tissue type following ischaemia in the upper extremity. Prolonged warm ischaemia leads to a cascade of deleterious biochemical and structural changes and, in conjunction with tissue and vascular damage, may inhibit or restrict successful reperfusion. Controlled hypothermia, as opposed to cryopreservation, has been shown to increase tolerance to ischaemia. Hypothermic preservation of amputated tissue is an established technique to maximize the possibility for successful replantation, although a poor cooling technique is likely to cause further cellular damage through either freezing or tissue maceration. Improved functional outcomes in rat models have been shown in limbs cooled to 22° C. and 30° C. (but not 35° C.) compared to normothermic equivalents (Gurke et al, 2000). Mowlavi et al (2003) also showed in the rat model that cooling is beneficial only during reperfusion rather than the entire ischaemic period. In other areas of clinical practice—in particular transplantation surgery—cool/cold ischaemic tissue preservation has become the norm, based on assessment of post-ischaemic tissue viability.

Ischaemia-Reperfusion (I-R) injury is a common complication for tourniquet-treated casualties with limb haemorrhage—the extent of this is related to the duration of tourniquet use (Klenerman, 1980; Klenerman, 2003; Kam et al, 2001; Blaisdell, 2002). Tissue temperature can significantly influence I-R injury, and limb cooling during ischaemia has been shown to reduce metabolic demand (Sapega et al, 1988) and inhibit microvascular and endothelial dysfunction (Schaser et al, 2006). The benefit of limb cooling has been shown clinically (Swanson et al, 1991; Ikemoto et al, 1988) and in experimental models with tourniquet application (Awerbuck et al, 1994; Fish et al, 1993) often reducing tissue temperature by 5-10%. Indeed, during the US Army experience in the Italian campaign of World War II, cold winter conditions were credited with increasing tourniquet toleration by up to 8 hours “without any apparent deleterious effect” (Wolff et al, 1945; Kragh et al, 2007).

However, there currently exists no convenient, portable and cost effective means of controlling the temperature of an injured limb which can be quickly and easily deployed and applied to a subject at or near the location where injury occurred, such as a battlefield.

It is an object of at least one embodiment of at least one aspect of the present invention to alleviate and/or mitigate one or more problems or disadvantages associated with the prior art.

### SUMMARY OF INVENTION

According to a first aspect of the present invention there is provided a tourniquet apparatus comprising:

at least two expandable members configured for applying pressure on a limb of a subject; and

actuating means for actuating the at least two expandable members,

wherein the actuating means are configured to actuate each expandable member independently and/or selectively.

The tourniquet apparatus may be or may comprise a tourniquet. A tourniquet may be defined as an apparatus capable of compressing a limb of a subject, for example in order to reduce, restrict or prevent blood flow in the region of the tourniquet. By such provision, in the event of an injury, the use of a tourniquet may reduce or may prevent bleeding through a wound and/or may reduce or may prevent external haemorrhage of the injured limb.

Typically, the apparatus may have two, e.g., a pair of, expandable members.

The apparatus may comprise a housing. The expandable members may be provided or may be located at least partially within the housing.

In use, the housing may define an opening configured for receiving a limb of a subject, e.g. a portion of an arm or a leg. In use, the housing may contact the limb of the subject.

In use, at least one expandable member, typically each expandable member, may define an opening configured for receiving a limb of a subject, e.g. a portion of an arm or a leg.

The housing may be rigid or flexible.

The expandable members may at least partially define the opening or may at least partially extend along an inner circumference or surface thereof.

The expandable members may be spaced apart, e.g. in a longitudinal direction or axis of the apparatus. In an embodiment, the expandable members may be spatially distal along an axis, e.g. a longitudinal axis, of the housing and/or opening. The expandable members may share a common axis, e.g. longitudinal axis, with the housing and/or opening.

The expandable members may be provided adjacent to each other or in proximity to each other. In an embodiment, the expandable members may be separated by a gap sufficient to allow actuation, e.g. inflation, of each expandable member without interference with another expandable member.

In an embodiment, the housing may be flexible, e.g. may be provided in the form of a flexible cuff or sleeve, which may for example be made of fabric, canvas, or the like. By such provision, the apparatus may be conveniently stored when not in use, and may be conveniently placed on a subject's limb when required. The housing may be made from a durable and/or hygienic material, e.g. from a polymeric material such as a PVC-coated polyester.

The housing may have at least one receiving portion or pocket for receiving one or more expandable members. Advantageously, the housing may have at least two receiving portions or pockets. The housing may have at least two receiving portions or pockets, each receiving portion or pocket configured to receive and/or secure a respective expandable member.

The expandable members may form part of the housing. The housing may contain or may define the expandable members.

The expandable members may have a stowed configuration and a deployed configuration. In use, in a stowed configuration, the expandable members may not engage the limb, may not apply pressure on the limb, and/or may not apply sufficient pressure on the limb so as to restrict blood flow. In a deployed configuration, the expandable members may be configured to engage the limb, to apply pressure on the limb, and/or to apply sufficient pressure on the limb so as to restrict blood flow.

One or more expandable members, typically each expandable member, may comprise or may be provided in the form of an inflatable member, such as an inflatable bladder. Thus, in a stowed configuration, the bladder(s) may be deflated, and in a deployed configuration, the bladder(s) may be inflated. In such instance the tourniquet apparatus may be defined as a pneumatic tourniquet.

The expandable members may be made from a material suitable to undergo repeated cycles of expansion and contraction, e.g. inflation and deflation. The expandable members may be made from a polymeric material such as a PVC-coated polyester.

Advantageously, each expandable member, e.g. bladder, may be actuated, e.g. inflated, independently.

Advantageously, in use, upon actuation of the apparatus, a first expandable member may be actuated, e.g., a first bladder may be inflated. The apparatus may be configured to allow actuation of a second expandable member, for example after a predetermined amount of time. By such provision, when a second expandable member is actuated from its stowed configuration to its deployed configuration so as to compress a subject's limb, the first expandable member may be disengaged from the subject's limb, e.g. may be moved from its deployed (inflated) configuration to its stowed (deflated) configuration. Advantageously, this allows the apparatus to maintain the desired level of compression on the limb and/or the desired level of blood flow restriction, while reducing the likelihood of local ischaemia and/or mechanical vascular and nerve damage at the point of application of the apparatus.

The apparatus may comprise fastening means for securing the apparatus, e.g. housing thereof, on a subject's limb. The fastening means may comprise conventional fastening mechanisms such as buttons, rivets, hook and loop fasteners (e.g., Velcro®) or the like.

Conveniently, the Velcro® fasteners may be provided on a surface of the housing, e.g., on a surface of the cuff or sleeve. Typically, one or more Velcro® strips (e.g. loops) may be provided on an outer surface of the cuff or sleeve, and a Velcro fastener (e.g. hooks) may be provided on a fastening portion of the apparatus typically located near at or near an end of the apparatus. By such provision, in use, the apparatus may be placed on a subject's limb so as to engage the limb, the fastening portion may be wrapped over a portion of the apparatus, e.g. of the housing, so as to bring the Velcro® fastening portion in contact with the one or more Velcro® strips, thus securing the apparatus on the subject's limb.

The housing, e.g. cuff or sleeve, may have at least two pockets, each pocket configured to receive and/or secure a respective bladder. In one embodiment, the housing may have two adjacent and substantially parallel pockets extending along a width of the cuff or sleeve, each pocket configured to receive a respective bladder.

The housing, e.g. cuff or sleeve, may define at least two bladders. In one embodiment, the housing may have or may define two adjacent and substantially parallel bladders extending along a width of the cuff or sleeve.

The bladders may be permanently attached to each other, e.g., may form an integral part of the housing. Alternatively, or additionally, one or more bladders may be attachable, e.g. via hook and loop fasteners, zips, ties, or the like, to another adjacent bladder. For example, in one embodiment, one or more bladders may be provided within a first housing which may also have a first cuff portion, and one or more bladders may be provided within a second housing which may also have a second cuff portion, the first housing and the second housing being detachably attachable to each other. By such provision, the gaps between two or more adjacent bladders may be adjusted, for example to suit the most favourable location of the bladders depending on the subject's injured limb and/or the location and size of the injuries.

Thus, in one embodiment, the present invention may relate to a tourniquet apparatus, e.g. a tourniquet, comprising:

a cuff or sleeve configured for engaging with a limb of a subject;

at least two inflatable bladders configured for applying pressure on a limb of a subject in an inflated configuration; and

actuating means for actuating the at least two inflatable bladders,

wherein the actuating means are configured to actuate each inflatable bladder independently and/or selectively.

In an embodiment, the actuating means may comprise pressure-inflating means, e.g. one or more pumps.

The apparatus may comprise or may be connectable to a plurality of pumps, each pump associated with a respective expandable member, e.g. bladder. Alternatively, the apparatus may comprise or may be connectable to a pump selectively associated with a plurality of expandable members, e.g. bladders. By such provision the apparatus may be actuated using a single pump, thus reducing costs and improving convenience, while allowing a user to selectably inflate each bladder.

The apparatus may comprise a control valve or switch configured to selectively actuate one or more expandable members, e.g. bladders, by the actuation means, e.g. pump.

One or more actuators, e.g. pumps, may be mechanically powered. In an embodiment, one or more actuators may comprise or may be a mechanical pump, e.g. a foot pump, a hand pump, or the like. By such provision, the tourniquet may be deployed and secured on a subject's limb without the need for external power, e.g. electrical power.

One or more actuators, e.g. pumps, may be externally powered, e.g., may be electrically powered. In such instance there may be provided a power supply, e.g. a portable power supply such as a battery, capable of powering one or more actuators, e.g. pumps and/or any accessory therefor, such as a compressor.

The control valve or switch may allow selective inflation of one or more bladders.

The control valve or switch may comprise or may be a valve having an inlet and a plurality of outlets. The inlet may be in communication with the actuator, e.g. pump, and may be configured for allowing a pressurised gas, e.g. air, into the valve. The valve may have a plurality of outlets, each outlet being in communication with an associated expandable member, e.g. bladder.

In an embodiment, the valve may have an inlet and two outlets, each outlet being in communication with a respective bladder.

In use, the valve may be controlled so as to be closed or open. When the valve is open, the valve may be configured to allow air to pass through at least one outlet.

In the open configuration, the valve may be configured to allow air to pass through a single outlet, e.g. a first outlet or a second outlet. By such provision, a user may selectable inflate a first bladder or a second bladder.

The valve may have a first open position in which the inlet is in fluid communication with the first outlet.

The valve may have a second open position in which the inlet is in fluid communication with the second outlet.

The valve may be configured such that, in a third or intermediate position, such as between the first open position and the second open position, the first outlet may be in fluid communication with the second outlet. In the third or intermediate position, such as between the first open position and the second open position, the inlet may not be in fluid communication with the first outlet or with the second outlet. By such provision, in use, pressure may be distributed between the first bladder and the second bladder, such that, when the valve is switched from a first open position to a second open position, the second bladder is already partially inflated and only requires a limited amount of additional pressure to reach its expanded configuration. This may ensure that, upon activation of the control valve or switching between a first bladder and a second bladder, sufficient pressure on the subject's limb is applied at all times. Advantageously, the momentary release of pressure from a bladder during gas transfer between the first bladder and a second bladder may allow momentary and/or controlled bleeding of the limb, which may help flush stagnant blood in the injured limb and avoid prolonged ischemia, thus improving the likelihood of preserving the injured limb.

The valve may have a dual outlet configuration in which the valve may allow air to pass through two or more outlets, e.g. two outlets, simultaneously, for example when the valve is moved from a first open position to a second open position. Thus, the valve may be configured such that, in a third or intermediate position, such as between the first open position and the second open position, the inlet is in fluid communication with the first outlet and the second outlet. By such provision, if a user wishes to “switch bladder”, for example after a predetermined amount of time, this may ensure that the second bladder is partially or fully inflated before the first bladder is allowed to deflate, thus ensuring that the primary function of the tourniquet is maintained at all times. This may help avoid undesirable release of pressure from one bladder before another bladder is fully inflated, for example in the event of the large injury causing extensive bleeding.

The apparatus may comprise a deflating mechanism to permit deflation of the bladders. The deflation mechanism may allow selective and/or independent deflation of each bladder.

The deflation mechanism may comprise one or more pressure release valves. Conveniently, the deflation mechanism may be provided within the control valve or switch. In an embodiment, the control valve or switch may have a plurality of pressure release valves, each pressure release valve being associated with a respective bladder in a predetermined position. For example, the control valve or switch may comprise a first pressure release valve associated with a first bladder and/or first outlet when the control valve or switch is in a second open position. By such provision the first bladder may be deflated via the first pressure release valve when the control valve or switch is in a second open position. The control valve or switch may comprise a second pressure release valve associated with a second bladder and/or second outlet when the control valve or switch is in a first open position. By such provision the second bladder may be deflated via the second pressure release valve when the control valve or switch is in a first open position.

A/each pressure release valve may be set to a predetermined release threshold, so as to allow deflation or partial deflation to a predetermined pressure. In one embodiment, a/each pressure release valve may be set of allow venting to atmospheric pressure. In other embodiment, a/each pressure release valve may be set of allow partial deflation to a predetermined pressure, e.g. 1.5 bar, 2 bar, or 3 bar.

The apparatus may comprise one or more sensors, e.g. pressure sensors or manometers. Typically, one or more sensors may be provided near the control switch or valve. A pressure sensor may be provided upstream of the control switch or valve, and/or may be configured to measure pressure associated with the inlet. One or more pressure sensors may be provided downstream of the control switch or valve, and/or may be configured to measure pressure associated with one or more outlets and/or bladders.

Provision of one or more pressure sensors, e.g. manometers, may allow a user to inflate the bladder(s) to a set pressure, for example to the limb occlusion pressure (LOP), which is commonly defined as the minimum pressure required to fully restrict arterial blood flow into a subject's limb. Typically, arterial pressure may be in the region of 90-140 mm Hg. The apparatus may be configured, e.g., programmed, to cause inflation of one or more bladders to a pressure above arterial pressure in deep tissue. Thus, the apparatus may be configured, e.g., programmed, to inflate one or more bladders to a pressure of at least 140 mm Hg, e.g., above 140 mm Hg, e.g. between 140 and 250 m Hg.

The pressure sensors and actuator(s) may be combined or may be provided in a single device, e.g. a sphygmomanometer.

One or more actuators, e.g. pumps, may be manually activated.

One or more actuators, e.g. pumps, may be automatically activated. In an embodiment, if the pressure detected by one or more pressure sensors is below a predetermined level, e.g. below the LOP, one or more actuators may be automatically activated. One or more pressure sensors may be associated with or may be in communication with one or more actuators. A controller may be associated with or in communication with one or more pressure sensors, and may activate one or more actuators, for example is pressure measured by one or more pressure sensors is below a predetermined level, e.g. below the LOP.

The control valve or switch may be manually operated.

The control valve or switch may be automatically operated. In such instance there may be provided a power supply, e.g. a portable power supply such as a battery, capable of powering the control valve or switch. In an embodiment, the control valve or switch may be controlled by a valve controller. The valve controller may be configured to switch the valve, e.g. from a first open position to a second open position (or vice versa), or from a closed position to a first open position or to a second open position (or vice versa), on a predetermined cycle, e.g. timed cycle. By such provision, inflation of each bladder may be cyclical and/or may follow a timed pattern such that inflation of each bladder lasts for a predetermined amount of time, and such that inflation alternates between bladders. This may help minimise the risks of located damage to a subject's tissue in the region of the tourniquet, and may reduce the risk of manual error associated with manually operating the control valve or switch.

According to a second aspect of the present invention there is provided a limb-cooling apparatus comprising:

an inner stretchable layer configured to engage and/or contact a limb of a subject;

an outer layer at least partially attached to the inner layer; and

a cooling system configured to supply a cooling medium between the inner layer and the outer layer.

The inner layer may be made of a stretchable fabric. The inner layer may be elastic. The inner layer may be made of a textile or fabric material, such as a polyester, polyurethane, copolymers thereof, blends or composites thereof, or any other suitable stretchable and/or elastic material. By such provision, the inner layer may be capable of snugly contacting a subject's limb and/or of ensuring a secure fit of the cooling apparatus to the subject's limb.

The inner layer may be thermally conductive. In an embodiment, the inner layer may be made from a polymer, e.g. a polyester-polyurethane copolymer such as Lycra®, Spandex, elastane, or the like. By such provision the inner layer may be capable of effectively conducting heat away from a subject's limb.

The outer layer may be or may form a thermally insulating layer. By such provision the cooling apparatus may be capable of containing the cooling medium, e.g. for an amount of time sufficient to allow cooling of the limb.

The outer layer may comprise or may be a polymeric layer. The outer layer may comprise or may be a metal film or foil. In an embodiment, the outer layer may comprise or may be a metal film or foil coated with a polymeric material. Advantageously, the metal film or foil may be provide on an inner side and/or on a side facing the inner layer, and the polymeric coating may be provided on an outer side and/or on a side facing away from the inner side. Such an arrangement may provide effective thermal insulation, e.g. via the metal film or foil, while providing adequate mechanical protection to the apparatus, e.g., via the polymeric coating. In an embodiment, the outer layer may comprise or may be an aluminium foil impregnated with a polymeric coating such as an acrylic, silicone, or polyurethane coating.

The outer layer and the inner layer may define at least one space therebetween. The outer layer and the inner layer may be attached so as to define the at least one space. In an embodiment, the outer layer and the inner layer may be attached at or near a peripheral region thereof and may define a space between the outer layer and the inner layer. The outer layer and the inner layer may be attached, e.g., stitched, fused or otherwise bonded together in or more regions distal from the periphery of the outer layer and/or of the inner layer. For example, the outer layer and the inner layer may be attached in a longitudinal direction thereof so as to define a plurality of channels between the inner layer and the outer layer. This may help a cooling medium supplied to the channels to remain within the channels, at least temporarily, thus providing effective cooling of the limb by avoiding the cooling medium to migrate to a localised area of the apparatus.

Typically, the inner layer and the outer layer may be flexible. This allows the cooling apparatus to be easily applied or affixed to the limb of a subject.

The cooling apparatus may be provided as a sock. In such instance the sock may be pulled onto a subject's limb. The sock may be secured to a subject's limb by the elastic nature of the inner layer itself, and/or by additional attachment means such as a band, strap, or the like.

The cooling apparatus may be provided as a sleeve. In such instance the sleeve may be wrapped onto a subject's limb. There may be provided fastening means for securing the cooling apparatus on a subject's limb. The fastening means may comprise conventional fastening mechanisms such as buttons, rivets, hook and loop fasteners (e.g., Velcro®) or the like. Conveniently, the fastening mechanism may comprise Velcro® fasteners. The Velcro® fasteners may be provided on a surface of the cooling apparatus. Alternatively, the Velcro® fasteners may be provided separately from the cooling apparatus. In an embodiment, the Velcro® fasteners may be provided as strips that may be wrapped over the cooling apparatus, e.g. sleeve, to secure the apparatus on the subject's limb.

The cooling medium may be a gas or a gas mixture. In one embodiment, the cooling medium may be carbon dioxide. In another embodiment, the cooling medium may be a mixture of a gas and a coolant fluid, for example a mixture of a gas, e.g. compressed air, and a halogenated coolant, e.g. a perfluorocarbon such as perfluorohexane.

The cooling system may comprise a delivery system for the cooling medium. The cooling system, e.g. delivery system, may comprise tubing configured for feeding the cooling medium between the inner layer and the outer layer.

The tubing may comprise a microporous tubing. By such provision, upon supply of a cooling medium under pressure, the cooling medium may permeate through the microporous material, thus expanding due to pressure difference and cooling as it expands. The tubing may be made of a microporous polymer, e.g. microporous PTFE (polytetrafluoroethylene). In an embodiment, the tubing may be made of ePTFE (expanded PTFE). ePTFE may provide advantageous properties in terms of mechanical strength, resilience, flexibility, and porosity.

The delivery system, e.g. tubing, may be connected to a container configured to supply the cooling medium. The container may comprise or may be a pressured canister and/or an aerosol canister.

The tubing may be provided between the first layer and the second layer. The tubing may be attached to or secured to a support layer configured to maintain the position and/or arrangement of the tubing, in use. The support layer may be flexible so as to maintain flexibility of the cooling apparatus, e.g. sleeve. The support layer may be provided in the form of a mesh, which may advantageously provide support to the tubing without interfering with heat transfer and/or flow of the cooling medium between the inner layer and the outer layer.

The apparatus may be provided with one or more temperature indicators. The temperature indicator(s) may provide information about the temperature in at least a region of the cooling apparatus. Conveniently, the temperature indicator(s) may be attached to, and/or may be provided on or within at least a portion of the outer layer, e.g. on an outer surface thereof. By such provision, a user may easily assess a change in temperature within the cooling apparatus, and may take appropriate action, e.g. inject more cooling medium into, or release cooling medium from, the cooling apparatus. The temperature indicator(s) may comprise or may be provided in the form of a temperature sensitive ink indicator. In an embodiment, the temperature indicator(s) may comprise a film or sheet attached to, e.g. bonded, glued, stitched, or laminated, to the outer layer, the film or sheet comprising one or more temperature sensitive inks. In another embodiment, the temperature indicator(s) may comprise temperature sensitive inks directly impregnated, dispersed within, coated on, or printed on the outer layer, e.g. on a surface thereof.

Advantageously, the cooling apparatus may be configured to be used in combination with the tourniquet apparatus according to the first aspect. By such provision, the entire system may provide not only an improved means of reducing or preventing haemorrhage, and may also prolong tolerance to ischemia in the subject's limb by providing controlled hypothermia, thus improving the chance of full or partial recovery to the subject's limb.

The cooling apparatus may be attached to the tourniquet apparatus according to the first aspect, e.g. by Velcro®, clips, straps, rivets, etc.

The cooling apparatus and the tourniquet apparatus may be provided as a combined or integrated system.

According to a third aspect of the present invention there is provided a limb-protecting apparatus comprising:

a flexible, gas-impermeable, material capable of containing a subject's limb; and

means for injecting a gas inside the material.

The material may comprise a membrane, film, fabric, or any other flexible material.

The material may be transparent. By such provision, the limb may be capable of protecting the subject's limb, while allowing a person to see the subject's limb.

The material may be attachable to a subject's limb, e.g. may be provided as a sock which can be secured to a subject's limb by a strap, Velcro®, band, or the like.

Advantageously, the limb-protecting apparatus may be configured to be used in combination with the tourniquet apparatus according to the first aspect, and/or with the cooling apparatus according to the second aspect. By such provision, the entire system may provide not only an improved means of reducing or preventing haemorrhage, and/or may prolong tolerance to ischemia in the subject's limb by providing controlled hypothermia, but may also protect the limb after application of the tourniquet and/or during hypothermic control of the limb.

In an embodiment, the limb-protecting apparatus may be attached to the tourniquet apparatus according to the first aspect, e.g. by Velcro®, clips, straps, rivets, etc.

In another embodiment, the limb-protecting apparatus may be attached to the limb cooling apparatus according to the second aspect, e.g. by Velcro®, clips, straps, rivets, etc.

The limp-protecting apparatus, cooling apparatus and tourniquet apparatus may be provided as a combined or integrated system.

The means for injecting a gas inside the material may comprise one or more pumps. Conveniently, the pump(s) may be the same pump(s) or may be one of the pumps used in combination with the apparatus according to a first aspect.

The pump(s) may be mechanically powered, e.g. may be a foot pump, a hand pump, or the like. By such provision, the limb-protecting apparatus may be deployed and secured on a subject's limb without the need for external power, e.g. electrical power.

The pump(s) may be externally powered, e.g., may be electrically powered. In such instance there may be provided a power supply, e.g. a portable power supply such as a battery, capable of powering one or more pumps, e.g. pumps and/or any accessory therefor, such as a compressor.

The system, e.g., the limb protecting apparatus, may have a cooling medium inlet to allow cooling medium to be delivered to the limb cooling apparatus through the limb protecting apparatus.

The system, e.g., the limb protecting apparatus, may have a cooling medium outlet to allow a fluid or gas to be released from the limb cooling apparatus through the limb protecting apparatus.

According to a fourth aspect of the present invention there is provided a system comprising an apparatus according to the first aspect of the present invention, an apparatus according to the second aspect, and/or an apparatus according to a third aspect.

When the system comprises all three apparatuses, the system may have the ability reduce or prevent haemorrhage, to prolong tolerance to ischemia in the subject's limb by providing controlled hypothermia, and to protect the limb.

According to a fifth aspect of the present invention there is provided a method of restricting blood flow to a subject's limb, comprising:

applying a tourniquet apparatus on the limb of a subject, the tourniquet apparatus comprising at least two expandable members;

actuating at least one of the expandable members.

Advantageously, the method may comprise actuating, e.g. inflating, each expandable member independently and/or selectively.

The method may comprise actuating, e.g. inflating, each expandable member alternately.

By such provision, when a second expandable member is actuated from a stowed configuration to a deployed configuration so as to compress a subject's limb, the first expandable member may be disengaged from the subject's limb, e.g. may be moved from a deployed configuration to a stowed configuration. Advantageously, this method allows the apparatus to maintain the desired level of compression on the limb and/or the desired level of blood flow restriction, while reducing the likelihood of local ischaemia and/or mechanical vascular and nerve damage at the point of application of the apparatus.

The features described in respect of the apparatus according to a first aspect may apply equally in respect of the method according the fifth aspect, and are not repeated here for brevity.

According to a sixth aspect of the present invention there is provided a method of cooling a subject's limb, comprising:

applying a limb-cooling apparatus on a subject's limb, the apparatus comprising an inner stretchable layer configured to engage and/or contact a limb of a subject, and an outer layer at least partially attached to the inner layer; and

supplying a cooling medium between the inner layer and the outer layer.

The features described in respect of the apparatus according to a second aspect may apply equally in respect of the method according the sixth aspect, and are not repeated here for brevity.

According to a seventh aspect of the present invention there is provided a method of protecting a subject's limb, comprising:

applying a limb-protecting apparatus on a subject's limb, the apparatus comprising a flexible, gas-impermeable, material capable of containing a subject's limb; and

injecting a gas inside the material.

The features described in respect of the apparatus according to a third aspect may apply equally in respect of the method according the seventh aspect, and are not repeated here for brevity.

### BRIEF DESCRIPTION OF DRAWINGS

Embodiments of the invention will now be given by way of example only, and with reference to the accompanying drawings, which are:

FIG. 1 a tourniquet apparatus according to a first embodiment of the present invention;

FIG. 2 a tourniquet apparatus according to a second embodiment of the present invention, before use;

FIG. 3 the apparatus of FIG. 2, in a deployed or “in use” configuration;

FIG. 4 a schematic view from above of a cuff of the apparatus of FIG. 2;

FIG. 5 a valve connector used in the apparatus of FIG. 2;

FIG. 6 components of the valve connector of FIG. 5;

FIG. 7 a perspective view of a control valve or switch for use in the apparatus of FIG. 1 or FIG. 2;

FIG. 8 an exploded view of the control valve or switch of FIG. 7;

FIG. 9 an elevated front view of the control valve or switch of FIG. 7 in a first open position;

FIG. 10 an elevated front view of the control valve or switch of FIG. 7 in a second open position;

FIG. 11 a view from above of an embodiment of a tourniquet apparatus according to the present invention, equipped with a hand pump;

FIG. 12 a view from above of another embodiment of a tourniquet apparatus according to the present invention, equipped with a foot pump;

FIG. 13 a perspective view of the tourniquet apparatus of FIG. 11, including Velcro® fasteners;

FIG. 14 a view from above of the tourniquet apparatus of FIG. 12, including Velcro® fasteners;

FIG. 15 a perspective view of the tourniquet apparatus of FIG. 13, in a folded configuration;

FIG. 16 a perspective view of the tourniquet apparatus of FIG. 13, applied on a limb of a subject;

FIG. 17 a perspective view of a limb-cooling apparatus according to an embodiment of the present invention;

FIG. 18 a view from above of the limb-cooling apparatus of FIG. 17, including a cooling medium delivery system;

FIG. 19 an enlarged view of a connection mechanism for connecting a cooling medium supply to the apparatus of FIG. 18;

FIG. 20 a view from a above of a tubing arrangement used in an alternative embodiment of a limb-cooling apparatus according to the present invention;

FIG. 21 a view from above of an embodiment of a thermal indicator used with the limb-cooling apparatus of the present invention;

FIG. 22 a limb-compressing and cooling system according to an embodiment of the present invention;

FIG. 23 a limb-compressing, cooling and protecting system according to an embodiment of the present invention.

### DETAILED DESCRIPTION OF DRAWINGS

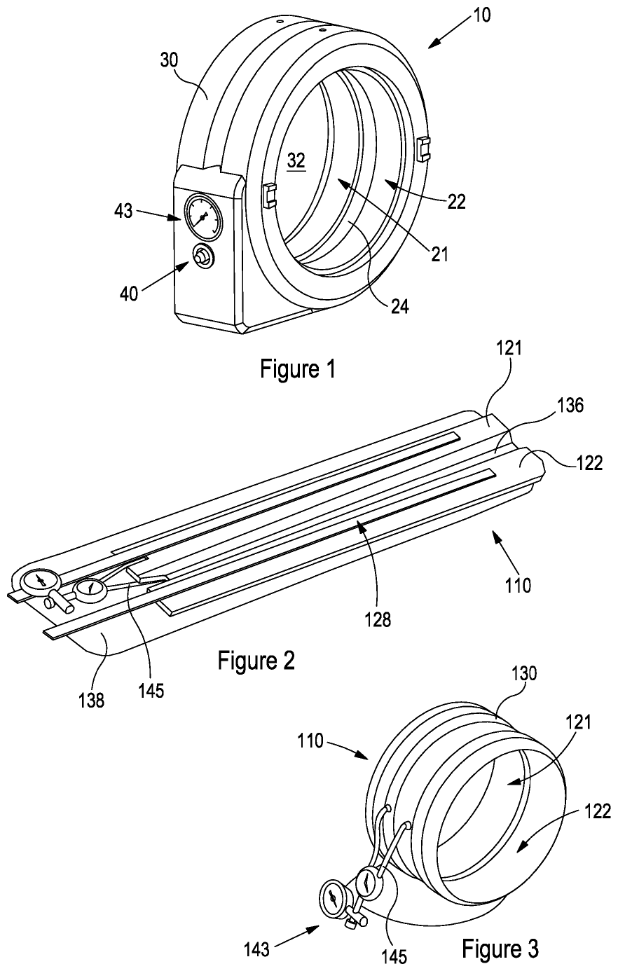

Referring to FIG. 1 there is shown a tourniquet apparatus, generally designated 10, according to a first embodiment of the present invention. The tourniquet apparatus 10 may be generally termed a tourniquet.

The tourniquet 10 comprises a pair of inflatable bladders 21,22, partially located within a housing 30. In this embodiment, the housing 30 is a rigid housing 30.

The housing defines an opening 32 for receiving a limb of a subject, e.g., a portion of an arm or a leg.

The bladders 21,22 are spatially arranged in a longitudinal direction along a axis of the housing 30 and opening 32. The bladders 21,22 share a common axis with the housing 30 and the opening 32.

The bladders 21,22 are provided adjacent to each other. In this embodiment, the bladders 21,22 are separated by a gap 24 having a size sufficient to allow inflation of each bladder without interference with the other bladder.

The housing 30 and/or opening 32 thereof is/are dimensioned so as to be capable of receiving a portion of a subject's limb. As such, the apparatus 10 is capable of receiving a subject's limb having a range of sizes, e.g., diameter. For example, the apparatus may be manufactured in a range of sizes, each size being designed to receive a predetermined range of limb sizes, for example corresponding to a typical region of a limb, e.g., a forearm, an arm, a lower leg (calf), or an upper leg (thigh).

The apparatus 10 has a gas inlet 40 configured to be connected to a pump (not shown) so as to independently and selectively inflate each bladder 21,22. The apparatus also has a pressure gauge or manometer 43 to monitor the pressure in the bladders.

Referring now to FIGS. 2 and 3, there is shown a tourniquet apparatus 110 according to a second embodiment of the present invention. The tourniquet 110 of FIG. 2 is similar to the tourniquet 10 of FIG. 1, like part denoted by like numerals, incremented by ‘100’.

The tourniquet 110 also has a housing 130 and a pair of bladders 121,122. However, in this embodiment, the housing 130 is a flexible housing 130. The housing 130 has an inflatable elongate portion 136 which has been heat-sealed along a length thereof in its central region so as to define two separate inflatable bladders 121,122. The inflatable bladders 121,122 are made a PVC-coated polyester material which allows the tourniquet 110 to undergo repeated cycles of expansion and contraction, e.g. inflation and deflation, without mechanical damage.

The tourniquet 110 also has a non-inflatable cuff portion 138 at an upper end of the bladders, arranged to affix and secure the tourniquet 110 on a subject's limb, as will be explained later in more detail.

FIG. 2 shows the tourniquet 110 in a non-inflated configuration, when not in use. FIG. 3 shows the tourniquet 110 in a deployed or “in use” inflated configuration. For illustration purposes, both inflatable bladders 121,122 are shown as inflated. However, the tourniquet 110 allows each bladder 121,122 to be inflated separately and/or independently, as will be explained later in more detail.

A manometer 143 is provided to measure pressure in the bladders 121,122.

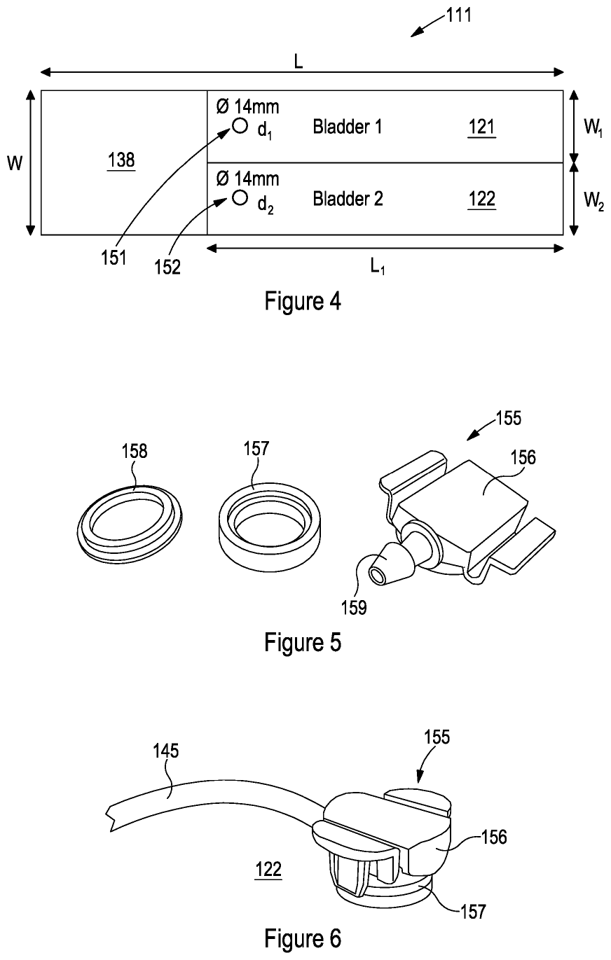

FIG. 4 shows a schematic view from above of a cuff structure 111 of the tourniquet 110 of FIG. 2. The tourniquet has a flexible housing 130 provided in the form of a cuff 111. The tourniquet 110 has an inflatable elongate portion 136 which has been heat-sealed along a length thereof in its central region so as to define two separate inflatable bladders 121,122. The tourniquet 110 also has a non-inflatable cuff portion 138 at an upper end of the bladders, arranged to affix and secure the tourniquet 110 on a subject's limb.

In this embodiment, the dimensions of the cuff 111 are selected to ensure that the tourniquet 110 is suitable for upper and lower limbs. As such, the cuff 110 was designed in accordance with dimensions stated by McDowell and colleagues in a 2008 National Statistics Report on Anthropometric Reference Data for Children and Adults in the United States between 2003-2006 (http://www.cdc.gov/nchs/data/nhsr/nhsr010.pdf), where the average adult mid-arm and mid-thigh circumferences were recorded as 39.4 cm and 55 cm, respectively.

Thus, in this embodiment, the cuff 111 had a total length L of 850 mm, and a total width W of 140 mm. The length L1 of the bladders was 600 mm, and the width W1,W2 of each bladder was 70 mm. The diameter d1,d2 of each port 151,152 for connecting to a pump inlet such as an inlet tube (not shown) was 14 mm.

Referring to FIGS. 5 and 6, there is shown a valve connector 155 used in the apparatus of FIG. 2 or with the cuff structure 111 of FIG. 4.

As shown in FIG. 5, the valve connector 155 provides a sealed connection between a tube 145 and a respective bladder 121,122. The valve connector includes a connector housing 156, which is sealably secured to port 152 of bladder 121,122 via first seal 157 and second seal 158. The connector housing 156 has a tube connector 159 to connected a tube 145, which tube 145 can be connected to a pump, manometer 143, or a valve as described later in more detail.

Where the tourniquet has a first bladder 121 and a second bladder 122, there may be provided a first valve connector 155a between a first tube 145a and a first bladder 121, and a second valve connector 155b between a second tube 145b and a second bladder 122.

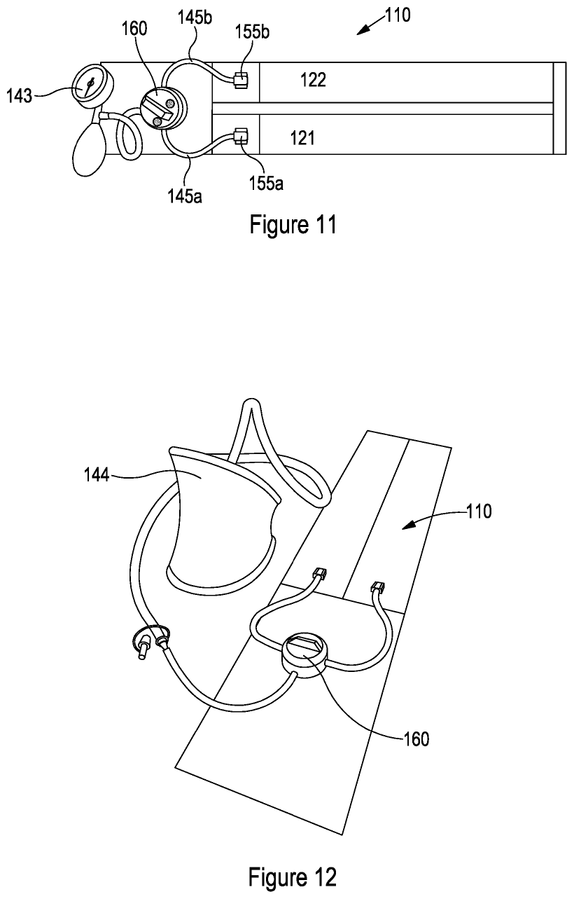

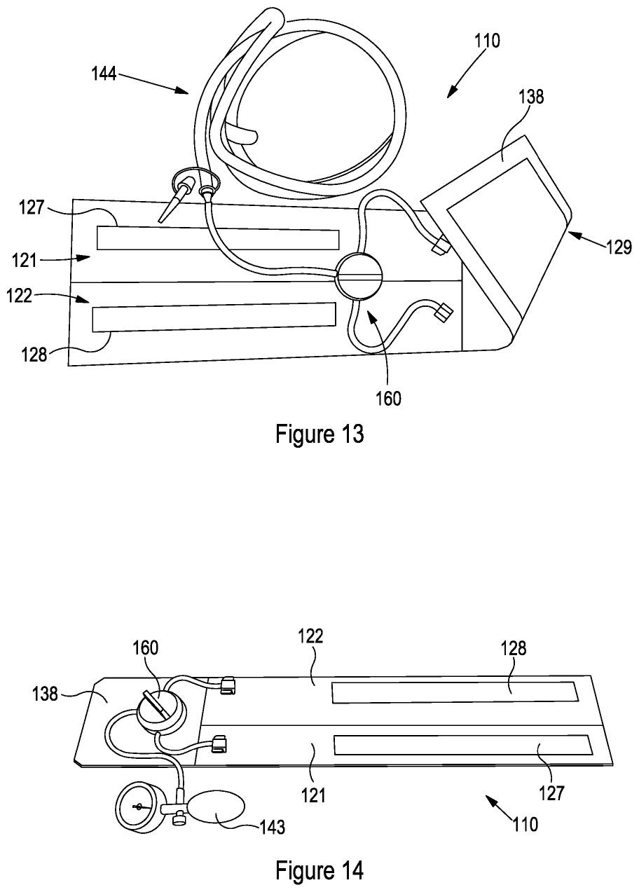

As shown in FIGS. 11 to 16, the tourniquet 110 is typically connected to one or more pumps capable of inflating bladders 121,122. In FIGS. 11, 14, 15 and 16, the pump is in the form of a sphygmomanometer 143, and in FIGS. 12 and 13 the pump is in the form of a foot pump 144. Although the present embodiments illustrate mechanically powered pumps, it will be appreciated that, in other embodiments, the pumps may be externally powered, e.g., may be electrically powered. In such instance there may be provided a power supply, e.g. a portable power supply such as a battery, capable of powering one or more actuators, e.g. pumps and/or any accessory therefor, such as a compressor.

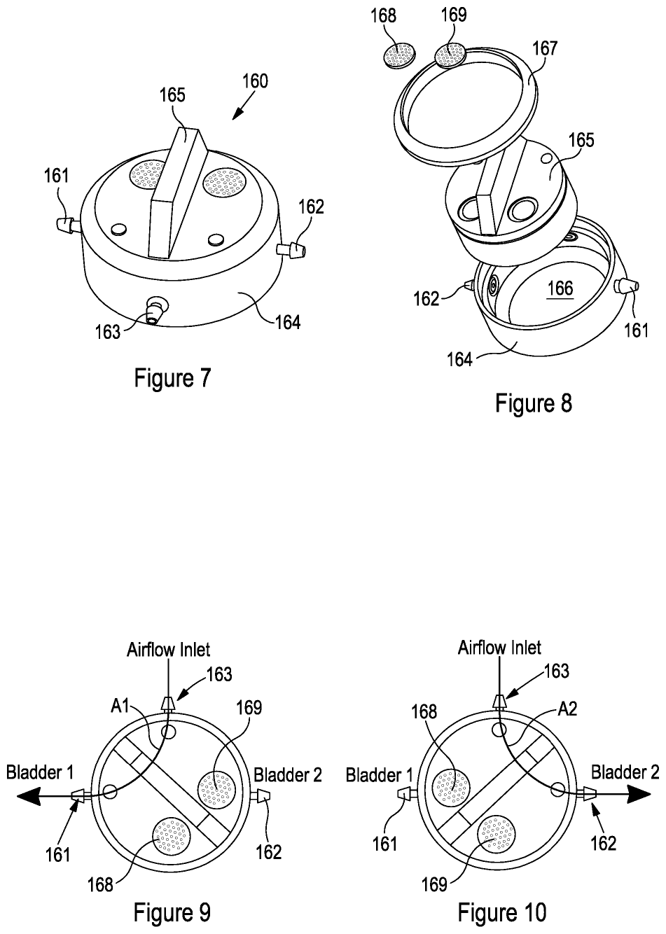

Advantageously, the bladders 121,122 are connected to the pump 143,144 via a control valve 160 as shown in FIG. 7. FIG. 8 shows an exploded view of the control valve 160 of FIG. 7. The control valve 160 is configured to selectively actuate of one or both bladders 121,122, by the associated pump 143,144. Thus, the control valve 160 allows selective inflation of one or both bladders 121,122.

The control valve 160 has an inlet 163 in communication with an associated pump 143,144. The control valve 160 has two outlets 161,162, each outlet being in communication with a respective bladder 121,122. It will be appreciated that, where the tourniquet 110 may have more than two bladders, the control valve may have more than two outlets, such that each outlet may be associated with a respective bladder.

In use, the valve may be controlled so as to be closed or open. When the valve is open, the valve may be configured to allow air to pass through at least one outlet.

As best shown in FIG. 8, the control valve 160 has a valve housing 164 which includes or is connected to inlet 163, first outlet 161 and second outlet 162. The control valve 160 also has a valve switch 165 which together with the valve housing 164 define a valve chamber 166 which is sealed by using a valve seal 167. The valve switch 165 is rotatable relative to the valve housing 164 so as to define a fluid passageway (now shown) between the inlet 163, first outlet 161 and/or second outlet 162.

The valve 160 is configured so as to allow fluid communication between the inlet 163 and the first outlet 161 in a first open configuration, as shown in FIG. 9. In this first open configuration, actuation of pump 143,144 inflates a first bladder 121. Thus, the valve switch 165 defines a fluid passageway represented by arrow A1, which, in the first open configuration, permits fluid communication between the inlet 163 and the first outlet 161.

The valve 160 is configured so as to allow fluid communication between the inlet 163 and the second outlet 162 in a second open configuration, as shown in FIG. 10. In this second open configuration, actuation of pump 143,144 inflates a second bladder 121. Thus, the valve switch 165 defines a fluid passageway represented by arrow A2, which, in the second open configuration, permits fluid communication between the inlet 163 and the second outlet 161.

The control valve 160 also has a deflating mechanism to permit deflation of the bladders 121,122, and in this embodiment to allow selective and/or independent deflation of each bladder 121,122.

The deflation mechanism includes pressure release valves 168,169. Which are be provided within the control valve 160. As best shown in FIGS. 9 and 10, the control valve 160 has two pressure release valves 168,169, each pressure release valve 168,169 being associated with a respective bladder 121,122 in a predetermined position. For example, as shown in FIG. 9, in the first open configuration, the second pressure valve 169 is in fluid communication with the second outlet 162 and thus acts as a vent for the second bladder 122. By such provision the second bladder 122 may be deflated via the second pressure release valve 169 when the control valve 160 is in a first open position. In contrast, as shown in FIG. 10, in the second open configuration, the first pressure valve 168 is in fluid communication with the first outlet 161 and thus acts as a vent for the first bladder 121. By such provision the first bladder 121 may be deflated via the first pressure release valve 168 when the control valve 160 is in a second open position.

In this embodiment, the valve 160 is configured such that, in an intermediate position between the first open position of FIG. 9 and the second open position of FIG. 10, the first outlet 161 is in fluid communication with the second outlet 162. By such provision, in use, pressure may be distributed between the first bladder 121 and the second bladder 122, such that, when the valve 160 is switched from a first open position to a second open position, the second bladder is already partially inflated and only requires a limited amount of additional pressure to reach its expanded configuration. Advantageously, the momentary release of pressure from the first bladder 121 during gas transfer between the first bladder 121 and a second bladder 122 may allow momentary and/or controlled bleeding of the limb, which may help flush stagnant blood in the injured limb and avoid prolonged ischemia, thus improving the likelihood of preserving the injured limb.

In alternative embodiments, the valve 160 may have a dual outlet configuration in which the valve 160 may allow air to pass through two or more outlets, e.g. two outlets, simultaneously, for example when the valve is moved from a first open position to a second open position. By such provision, if a user wishes to “switch bladder”, for example after a predetermined amount of time, this may ensure that the second bladder 122 is partially or fully inflated before the first bladder 121 is allowed to deflate, thus ensuring that the primary function of the tourniquet is maintained at all times.

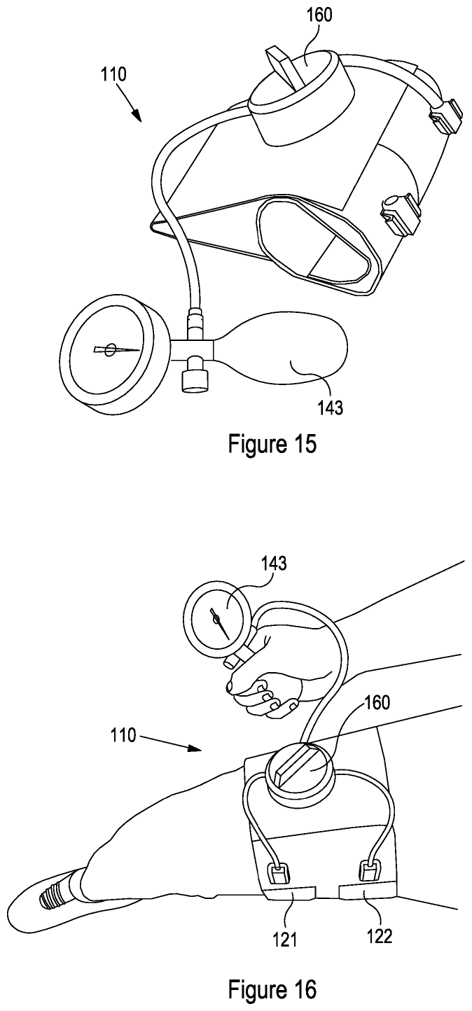

As best shown in FIGS. 13 and 14, the tourniquet 110 has fastening means 125 for securing the tourniquet 110 on a subject's limb, as for example a subject's leg as shown in FIG. 16. In this embodiment, the fastening means 125 include hook and loop fasteners 126, e.g., Velcro®. Conveniently, Velcro® strips (e.g. loops) 127,128 have been provided on an outer surface of the cuff 111, and in this embodiment on an outer surface of the first bladder 121 and second bladder 122. A Velcro fastener (e.g. hooks) 129 has provided on a cuff portion 138 of the tourniquet 110. By such provision, in use, the tourniquet 110 may be placed on a subject's limb so as to engage the limb, the cuff portion 138 may be wrapped over and secured to the bladders 121,122 so as to bring the Velcro® fastener 129 in contact with the Velcro® strips 127,128, thus securing the apparatus 110 on the subject's limb.

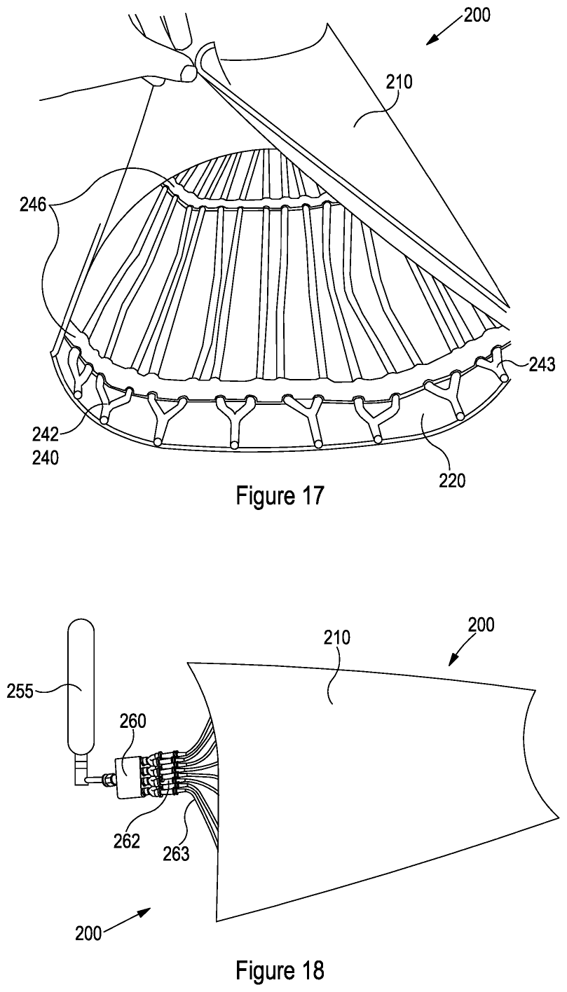

Referring to FIGS. 17-21, there is shown a limb-cooling apparatus, generally designated 200, according to an embodiment of the present invention.

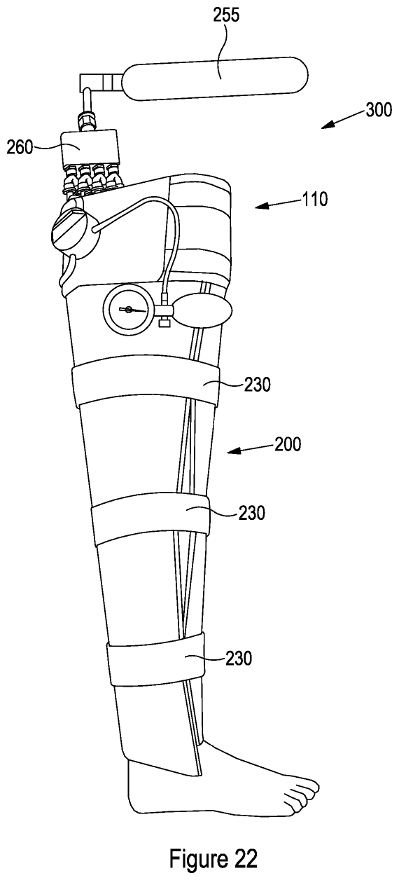

The limb-cooling apparatus 200 will be herein described in the context of a system 300 as described in FIG. 22, which also contains a tourniquet 310 which is similar to the tourniquet 110 as described with reference to FIGS. 2-16. The limb-cooling apparatus 200 will also be herein described in the context of a system 400 as described in FIG. 23, which contains a tourniquet 410, for example as described in FIG. 1, a limb-cooling apparatus 200, and a protecting system 490.

The limb-cooling apparatus 200 has an inner stretchable layer 210 configured to engage and/or contact a limb of a subject. In this embodiment, the inner layer 210 is made of a stretchable, elastic, fabric. Thus, the inner layer 210 is capable of snugly contacting a subject's limb and/or of ensuring a secure fit of the cooling apparatus 200 on the subject's limb.

Preferably, the inner layer 210 is also thermally conductive. By such provision the inner layer 210 is capable of effectively conducting heat away from a subject's limb so as to facilitate cooling.

In the present embodiment, the inner layer 210 is made from 130 g polyester-polyurethane copolymer marketed under the name UnderArmour® (UnderArmour Europe B.V., Amsterdam, Netherlands).

The limb-cooling apparatus 200 also has an outer layer 220 at least partially attached to the inner layer 210.

The outer layer 220 preferably forms a thermally insulating layer. By such provision the cooling apparatus 200 may be capable of containing a cooling medium between the inner layer 210 and the outer layer 220, e.g. for an amount of time sufficient to allow cooling of the limb.

In the present embodiment, the outer layer was made from Shieldtex/780®, a heat shield fabric made of a 20-micron aluminium foil impregnated with a black flame resistant polyurethane coating (Textile Technologies, Cheshire, UK).

The outer layer 220 and the inner layer 210 define a space 215 therebetween. As best shown in FIG. 17, the outer layer 220 and the inner layer 210 are attached at or near a peripheral region thereof so as to define a space 215 therebetween. The outer layer 220 and the inner layer 210 may be stitched, fused or otherwise bonded together.

In certain embodiments, the outer layer 220 and the inner layer 210 may be further attached in or more regions distal from the periphery of the outer layer and/or of the inner layer. For example, the outer layer 220 and the inner layer 210 may be attached in a longitudinal direction thereof so as to define a plurality of channels 217 between the inner layer 210 and the outer layer 220, as shown in FIG. 23. This may help a cooling medium 250 supplied to the channels 217 to remain within the channels 217, at least temporarily, thus providing effective cooling of the limb by avoiding the cooling medium to migrate to a localised area of the apparatus.

Advantageously, the inner layer 210 and the outer layer 220 are flexible, which allows the cooling apparatus 200 to be easily applied or affixed to the limb of a subject.

In the embodiments of FIGS. 17-22, the cooling apparatus 200 is provided as a sleeve which is configured to be wrapped onto a subject's limb. As shown in FIG. 22, there are provided Velcro® fasteners 230 in the form of strips so as to secure the apparatus 200 on the subject's limb.

In other embodiments, the cooling apparatus 200 may be provided as a sock. In such instance the sock may be pulled onto a subject's limb. The sock may be secured to a subject's limb by the elastic nature of the inner layer itself, and/or by additional attachment means such as a band, strap, or the like.

The limb-cooling apparatus 200 also includes a cooling system 240 configured to supply a cooling medium 250 between the inner layer 210 and the outer layer 220.

The cooling medium 250 may be a gas or a gas mixture. In one embodiment, the cooling medium may be carbon dioxide. In another embodiment, the cooling medium may be a mixture of a gas and a coolant fluid, for example a mixture of a gas, e.g. compressed air, and a halogenated coolant, e.g. a perfluorocarbon such as perfluorohexane. The cooling medium 250 is stored and delivered from a canister 255, as shown in FIGS. 18 and 22.

The cooling system 240 comprises a delivery system 242 for the cooling medium 250. The delivery system 242 includes tubing 243 configured to deliver the cooling medium 250 in the space 215 between the inner layer 210 and the outer layer 220. Advantageously, the tubing 243 is a microporous tubing. By such provision, upon supply of a cooling medium 250 under pressure, the cooling medium 250 can permeate through the microporous material, thus expanding due to pressure difference and cooling as it expands. The tubing 243 is preferably made from a microporous polymer such as microporous ePTFE (expanded polytetrafluoroethylene). ePTFE may provide advantageous properties in terms of mechanical strength, resilience, flexibility, and porosity.

The delivery system 242, e.g. tubing 243, is connected to the container 255 configured to supply the cooling medium 250.

As explained above, the tubing 243 is provided between the first layer 210 and the second layer 220. As shown in FIG. 20, the tubing 243 may be attached to or secured to a support layer 245 configured to maintain the position and/or arrangement of the tubing 240, in use. The support layer 245 is flexible so as to maintain flexibility of the cooling apparatus 200. In the embodiment of FIG. 20, the support layer 245 is provided in the form of a mesh, which advantageously provides support to the tubing 240 without interfering with heat transfer and/or flow of the cooling medium 250 between the inner layer 210 and the outer layer 220.

Alternatively, as shown in FIG. 17, the tubing 243 may be attached and/or connected to strips 246 so as to maintain the arrangement, configuration, and/or position of the tubing 243 between the first layer 210 and the second layer 220.

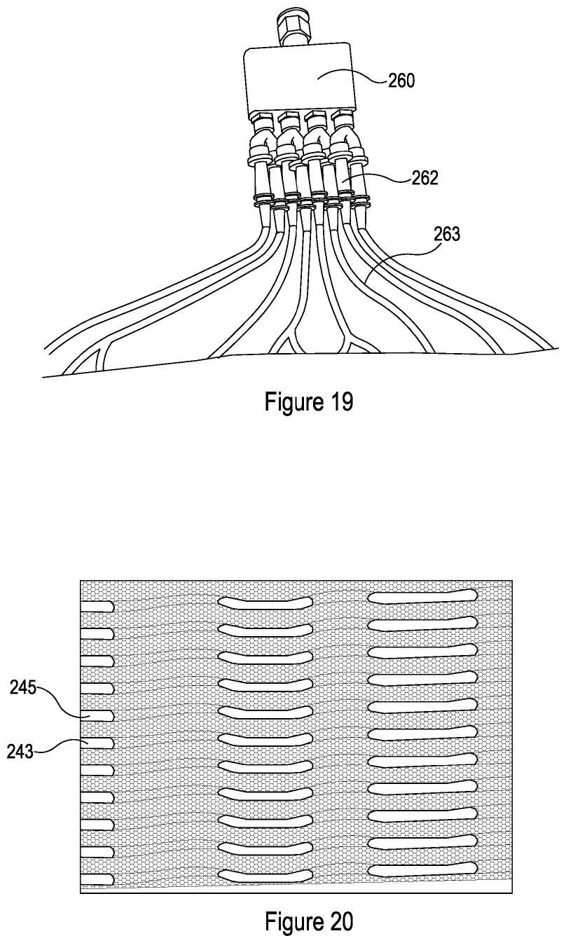

As best shown in FIGS. 18 and 19, the cooling apparatus 200 includes a manifold 260 with an inlet 261 connected to the container 255, and multiple outlets 262 each connected to a respective outlet tube 263 to supply cooling medium 250 to the tubing 243. By such provision, cooling medium may be evenly supplied to the tubing 243 so as to permit even cooling of the space 215 between the inner layer 210 and the outer layer 220.

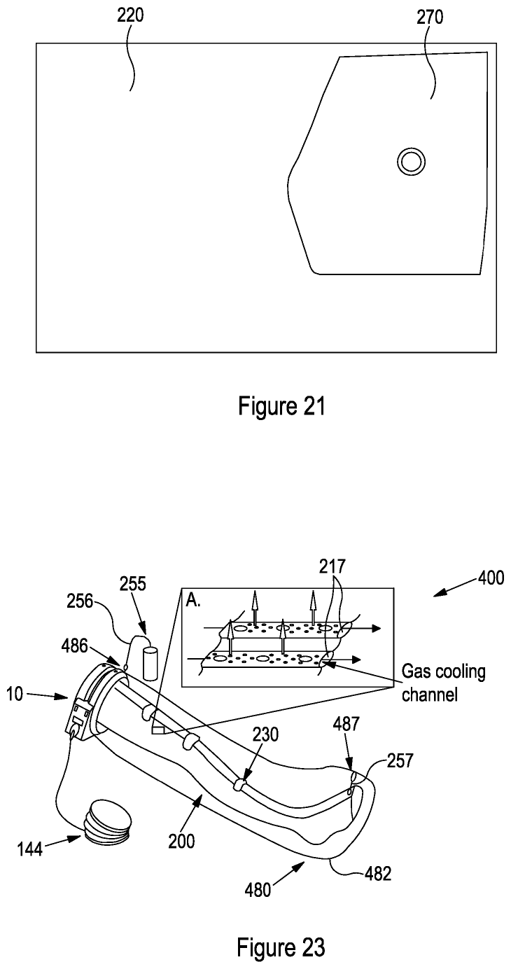

As best shown in FIG. 21, the apparatus 200 is also provided with one or more temperature indicators 270. The temperature indicator(s) 270 provide information about the temperature in at least a region of the cooling apparatus 200. In this embodiment, the temperature indicator 270 is attached to an outer surface of the outer layer 220. By such provision, a user may easily assess a change in temperature within the cooling apparatus 200, and may take appropriate action, e.g. inject more cooling medium 250 into, or release cooling medium 250 from, the cooling apparatus 200.

In this embodiment, the temperature indicator 270 is provided in the form of a temperature sensitive ink indicator within a film bonded or laminated, to the outer layer 220. In another embodiment, the temperature indicator(s) 270 may comprise temperature sensitive inks directly impregnated, dispersed within, coated on, or printed on the outer layer 220, e.g. on a surface thereof.

Advantageously, as shown in FIG. 22, the cooling apparatus 200 can be used in combination with the tourniquet 110 of FIGS. 2-16. By such provision, the entire system 300 may provide not only an improved means of reducing or preventing haemorrhage, and may also prolong tolerance to ischemia in the subject's limb by providing controlled hypothermia, thus improving the chance of full or partial recovery to the subject's limb.

In this embodiment, the tourniquet 110 and provided adjacent, and slightly overlapping, the cooling apparatus 200. It will be appreciated that, in other embodiments, the tourniquet 110 may be attached to each other, for example to reduce movement relative to each other and/or a patient's limb, for example by Velcro®, clips, straps, rivets, etc.

Referring to FIG. 23, there is shown a tissue preservation system 400 according to an embodiment of the invention.

The tissue preservation system 400 includes a tourniquet 10 as described with reference to FIG. 1. It will be appreciated that the tourniquet used in the system 400, in other embodiments, may be a tourniquet 10 as described with reference to FIG. 1.

The system 400 also includes a cooling apparatus 200 as described with reference to FIGS. 17-21.

The system also includes a limb-protecting apparatus 480 which has a flexible, gas-impermeable, material 482 capable of containing a subject's limb, in this embodiment, a leg. The limb-protecting apparatus 480 also has a means 484 for injecting a gas inside the material 482, which in this embodiment is the foot pump which is also used to inflate the tourniquet 10.

The material 482 is made of a flexible transparent membrane which can be inflated and is sufficient strong and resilient to protect the limb, and also allows a user, e.g. an attendant or clinician, to see the subject's limb.

The limb-protecting apparatus 480 is attachable to the tourniquet 10, which allows the various elements 10,200,480 to be used as a entire or integrated system 400. By such provision, the entire system 400 may provide not only an improved means of reducing or preventing haemorrhage, and/or may prolong tolerance to ischemia in the subject's limb by providing controlled hypothermia, but may also protect the limb after application of the tourniquet 10 and/or during hypothermic control of the limb.

In other embodiments, the limb-protecting apparatus 480 may be attached to the limb cooling apparatus 200 rather than, or in addition to, the tourniquet 10.

The limb-protecting apparatus 480 also has an inlet valve or port 486 to allow a tube 256 connected to the canister 255 to pass through the limb-protecting apparatus 480 and connect to the cooling system 240 of the limb-cooling apparatus 200. The limb-protecting apparatus 480 also has an outlet valve or port 487 to allow a tube 257 to release cooling medium from the limb-cooling apparatus 200 when required.

It will be appreciated that the embodiments of the invention hereinbefore described are given by way of example only and are not meant to limit the scope thereof in any way.

1. A tourniquet apparatus comprising:
at least two expandable members configured for applying pressure on a limb of a subject; and
an actuator coupled to the at least two expandable members,
wherein the actuator is configured to actuate to inflate each expandable member independently and/or selectively.

2. A tourniquet apparatus according to claim 1, comprising a pair of expandable members as the at least two expandable members.

3. A tourniquet apparatus according to claim 1, wherein the apparatus comprises a housing, the expandable members being provided or located at least partially within the housing.

4.-5. (canceled)

6. A tourniquet apparatus according to claim 3, wherein the expandable members at least partially extend along an inner circumference or surface of the housing and are spaced apart in a longitudinal direction or axis of the apparatus.

7. (canceled)

8. A tourniquet apparatus according to claim 1, wherein each of the at least two expandable members is inflatable independently.

9. A tourniquet apparatus according to claim 1, wherein the at least two expandable members comprise first and second expandable members, wherein the apparatus is configured such that when the second expandable member is actuated from a deflated stowed configuration to an inflated configuration so as to compress a subject's limb, the first expandable member is moved from an inflated configuration to a deflated configuration.

10. (canceled)

11. A tourniquet apparatus according to claim 1, wherein the apparatus comprises a control valve or switch coupled to the actuator and configured to selectively inflate one or more of the at least two expandable members.

12. A tourniquet apparatus according to claim 11, wherein the control valve or switch has an inlet in communication with the actuator, and a plurality of outlets comprising at least a first outlet and a second outlet, each outlet being in communication with an associated expandable member of the at least two expandable members, wherein the control valve or switch has a first open position in which the inlet is in fluid communication with the first outlet, and a second open position in which the inlet is in fluid communication with the second outlet.

13. (canceled)

14. A tourniquet apparatus according to claim 12, wherein the control valve or switch is configured such that, in a third or intermediate position, the first outlet is in fluid communication with the second outlet.

15. A tourniquet apparatus according to claim 12, wherein the control valve or switch is configured such that, in a third or intermediate position, the inlet is in fluid communication with the first outlet and the second outlet.

16. A tourniquet apparatus according to claim 11, wherein the at least two expandable members comprise first and second expandable members, wherein the apparatus further comprises a deflating mechanism to allow selective and/or independent deflation of the expandable members, wherein the control valve or switch comprises the deflation mechanism, the control valve or switch having a first pressure release valve associated with the first expandable member and/or a first outlet when the control valve or switch is in a second open position, and a second pressure release valve associated with the second expandable member and/or a second outlet when the control valve or switch is in a first open position.

17. (canceled)

18. A tourniquet apparatus comprising:
a cuff or sleeve configured for engaging with a limb of a subject;
at least two inflatable bladders configured for applying pressure on the limb of the subject in an inflated configuration; and
an actuator coupled to the at least two inflatable bladders,
wherein the actuator is configured to actuate each inflatable bladder independently and/or selectively.

19. A limb-cooling apparatus comprising:
an inner stretchable layer configured to engage and/or contact a limb of a subject;
an outer layer at least partially attached to the inner layer; and
a cooling system configured to supply a cooling medium between the inner layer and the outer layer.

20. A limb-cooling apparatus according to claim 19, wherein the inner layer is made of a stretchable fabric, and wherein the inner layer is thermally conductive.

21. (canceled)

22. A limb-cooling apparatus according to claim 19, wherein the outer layer is a thermally insulating layer.

23. A limb-cooling apparatus according to claim 19, wherein the outer layer and the inner layer define at least one space therebetween, wherein the outer layer and the inner layer are attached in a longitudinal direction so as to define a plurality of channels between the inner layer and the outer layer.

24.-25. (canceled)

26. A limb-cooling apparatus according to claim 19, wherein the cooling system comprises a delivery system for the cooling medium, the delivery system comprising tubing configured for feeding the cooling medium between the inner layer and the outer layer, wherein the tubing comprises a microporous tubing, wherein the tubing is attached to or secured to a support layer configured to maintain the position and/or arrangement of the tubing, in use.

27.-28. (canceled)

29. A limb-cooling apparatus according to claim 19, wherein the apparatus comprises one or more temperature indicators attached to and/or provided on or within at least a portion of the outer layer.

30.-32. (canceled)

33. A system comprising:
a tourniquet apparatus comprising:
at least two expandable members configured for applying pressure on a limb of a subject; and
an actuator coupled to the at least two expandable members,
wherein the actuator is configured to actuate to inflate each expandable member independently and/or selectively; and
a limb-cooling apparatus comprising:
an inner stretchable layer configured to engage and/or contact the limb of the subject;
an outer layer at least partially attached to the inner layer; and
a cooling system configured to supply a cooling medium between the inner layer and the outer layer.

34. A method of restricting blood flow to a subject's limb, comprising:
applying a tourniquet apparatus on the limb of a subject, the tourniquet apparatus comprising at least two expandable members; and
actuating each expandable member independently and/or selectively.

35. The method of claim 34, comprising actuating each expandable member alternately.

36. A method of cooling a subject's limb, comprising:
applying a limb-cooling apparatus on a subject's limb, the apparatus comprising an inner stretchable layer configured to engage and/or contact a limb of a subject, and an outer layer at least partially attached to the inner layer; and
supplying a cooling medium between the inner layer and the outer layer.

37. (canceled)
